(19) United States
(12) Patent Application Publication
(10) Pub. No.: US 2011/0301695 A1
(43) Pub. Date: Dec. 8, 2011

(54) OPTIMUM COATINGS FOR VASCULAR STENTS

(75) Inventor: ROBERT E. FISCHELL, DAYTON, MD (US)

(73) Assignee: SVELTE MEDICAL SYSTEMS, INC., NEW PROVIDENCE, NJ (US)

(21) Appl. No.: 12/795,712

(22) Filed: Jun. 8, 2010

(51) Int. Cl. A61F 2/82

(52) U.S. Cl. 623/1.46

(57) ABSTRACT

Disclosed is a stent that allows rapid coverage of the stent's luminal surface with endothelial cells while eluting enough anti-restenosis drug from the stent's abluminal surface to eliminate restenosis. A delay in the release of the anti-restenosis drug from the abluminal surface of the stent struts is created by a drug-free biodegradable polymer that covers that abluminal surface of the stent The anti-restenosis drug being only on the abluminal surface of the stent and the release of that drug being delayed by the outer polymer covering of that abluminal surface allows endothelial cells to have unconstrained mitosis so that they quickly cover the stent's luminal surface. It is further conceived to cover the luminal surface of the stent with an anti-thrombogenic coating such as carbon to further encourage endothelial cell coverage while deterring the deposition of platelets.

### FIELD OF USE

This invention is in the field of methods and devices for stenting of blood vessels in the body of human subjects, especially for stent implantation into peripheral and coronary arteries.

### BACKGROUND OF THE INVENTION

For many years, stents have been used to open stenosed arteries. The first such products were bare metal stents that were formed from stainless steel. More recent stent structures are made from higher density metal alloys such as L605, which is a cobalt-chromium alloy. Most modern drug eluting stents are now coated with a polymer containing a drug that elutes into the artery wall to prevent cellular proliferation that can cause restenosis of that artery. It is also well known to coat the stent surface with a material to decrease stent thrombosis, i.e., the formation of a blood clot in the region where the stent is implanted into the artery. One such coating is carbon and another example of an anti-thrombogenic coating is called Hepacoat, which coating includes the anti-thrombogenic drug heparin.

To prevent stent thrombosis, it is desirable to promote the adherence of endothelial cells onto the surface of the stent struts that are in contact with the blood flow through the lumen of the artery. The surface of the stent struts that face the lumen of the artery where the blood flows is called the “luminal” surface. The opposite side of the stent strut, which side is in contact with the arterial wall, is called the “abluminal” surface. To prevent stent thrombosis it is also desirable to prevent platelet deposition onto the luminal surface of the stent.

It is well known to have a stent coating that includes a drug to elute into the arterial wall to inhibit mitosis of cells that would proliferate due to the damage to the arterial wall from the expansion of the stent into that arterial wall. One problem with such anti-proliferative coatings is that they also inhibit the proliferation of endothelial cells onto the luminal surface of the stent, which endothelial cells are needed to prevent the formation of blood clots on the stent's luminal surface. Thus, what is a good drug effect to prevent restenosis is a bad effect in that it can inhibit the growth of endothelial cells onto the luminal surface of the stent struts.

It is also known that most of the cells that proliferate from the arterial wall maximize their rate of proliferation many days after stent implantation. If the release of the anti-proliferative drug could be delayed so that the endothelial cells could first start forming onto the luminal surface of the stent struts and then the anti-proliferative drug would be released at a later time to decrease cellular proliferation from the arterial wall, that would be an optimum stent coating design. In that way, the endothelial cells would first begin to coat the luminal side of the stent and then the drug would be released into the arterial wall to prevent restenosis.

Another important aspect of drug eluting stents is their shelf life. For example, the Cypher drug eluting stent (Cordis Corporation) has a shelf life in the USA of only 90 days. This results in a large fraction of these stents that are held in inventory at many hospitals being returned to the manufacturer because they become out-of-date. Any method that could be used to prolong stent shelf life would be very advantageous for the company that sells such a stent.

### SUMMARY OF THE INVENTION

The main goal of the present invention is to have a stent that allows rapid coverage of the stent's luminal surface with endothelial cells while eluting enough anti-restenosis drug from the stent's abluminal surface to eliminate restenosis. Furthermore, a goal of the present invention is to delay the release of the anti-restenosis drug from the abluminal surface of the stent struts so that the endothelial cells do not have their mitosis slowed down by contact with the anti-restenosis drug which is a drug that is designed to prevent cellular mitosis. Still further, an optimum design for the stent would have a roughened surface on all surfaces of the stent. Such a structure on the stent's luminal surface tends to discourage the deposition of platelets and encourage the growth of endothelial cells, thereby promoting the anti-thombogenic nature of the stent's luminal surface. Also, a roughened surface on the stent's abluminal surface could contain the drug to be eluted without the use of a polymer. Placing the drug into a roughened metal abluminal surface instead of placing the drug into a polymer would provide the desired attribute of a long shelf-life for the drug. Finally, a biodegradable polymer placed over the drug contained within the roughened abluminal surface of the stent, could delay the release of the drug into the artery wall for a few days, which does not adversely affect restenosis but would allow rapid endothelial cell growth onto the stent's luminal surface. Such a time delay would optimize the mitosis of the endothelial cells that are needed to cover the luminal side of the stent so as to prevent the formation of blood clots on the stent's luminal surface. Thus the three goals of no restenosis, no stent thrombosis and an extended shelf-life for the stent could be achieved.

One important inventive concept of the present invention is to place the anti-restenosis drug only on the abluminal surface of the stent struts. This feature alone would allow more rapid covering of the luminal surface of the stent with endothelial cells. Still further, the present invention envisions placing an anti-thrombogenic surface on the luminal surface of the struts such as carbon or Hepacoat. The present invention envisions placing a coating on the abluminal surface of each strut that that elutes the anti-restenosis drug, which coating is coated with a second coating that is a biodegradable or bioabsorbable coating that delays the release of the anti-restenosis drug into the cells of the arterial wall and delays its release onto the artery's endothelial cells.

Another extremely important advantage of the stent coatings as described herein is that they would allow reduced usage of anti-thrombogenic drugs such as Plavix. At this time, because of the tendency of stents to cause blood clots, the use of Plavix is frequently prescribed for the rest of the patient's life. However, if surgery is required, the patient must cease the use of such anti-thrombogenic drugs because they can cause uncontrolled bleeding. Still further, many patients have an adverse reaction to Plavix. Furthermore, the annual cost of Plavix in the USA is over a $1,000 which is a financial burden on the patient and on the health care system. If the coatings as described herein are placed onto the surfaces of the stent struts, then the use of Plavix could be limited to one or two months or possibly never used at all.

Thus one object of the present invention is to have only the abluminal surface of the stent coated with an anti-restenosis drug that elutes with a time delay of at least a few days.

Another object of this invention is to have the luminal surface of the stent be covered with a material and/or with a roughened surface finish that favors the deposition of endothelial cells and minimizes platelet deposition.

Still another object of this invention is to have a stent that has a roughened surface on the abluminal surface of the stent struts, which roughened surface contains the drug to be eluted. This design having an extended shelf-life for the stent.

Still another object of this invention is to have a stent that does not require extended use of an anti-thrombogenic drug such as Plavix in order to prevent the formation of blood clots in the region of the stent. The stent's luminal surface being covered with an anti-thrombogenic material such as carbon or Hepacoat.

These and other objects and advantages of this invention will become obvious to a person of ordinary skill in this art upon reading the detailed description of this invention including the associated drawing as presented herein.

### BRIEF DESCRIPTION OF THE DRAWINGS

FIG. 1 is a cross section of a single strut of a stent, which strut is coated on its abluminal surface with an anti-restenosis drug placed within a polymer, that polymer being coated with a second polymer coating that delays the release of the drug into the tissue of the arterial wall, the strut also having a luminal side that enhances the deposition of endothelial cells while generally rejecting the deposition of platelets onto that luminal surface of the strut.

FIG. 2 is a cross section of a single strut of a stent, which strut has roughened surfaces on both its luminal and abluminal surfaces with a restenosis drug contained within the roughened metal abluminal surface and that abluminal surface having an exterior biodegradable polymer coating that provides for the delayed release of the anti-restenosis drug into the tissue of the arterial wall, the strut also having a luminal surface that enhances the deposition of endothelial cells while generally rejecting the deposition of platelets onto that luminal surface of the strut.

### DETAILED DESCRIPTION OF THE INVENTION

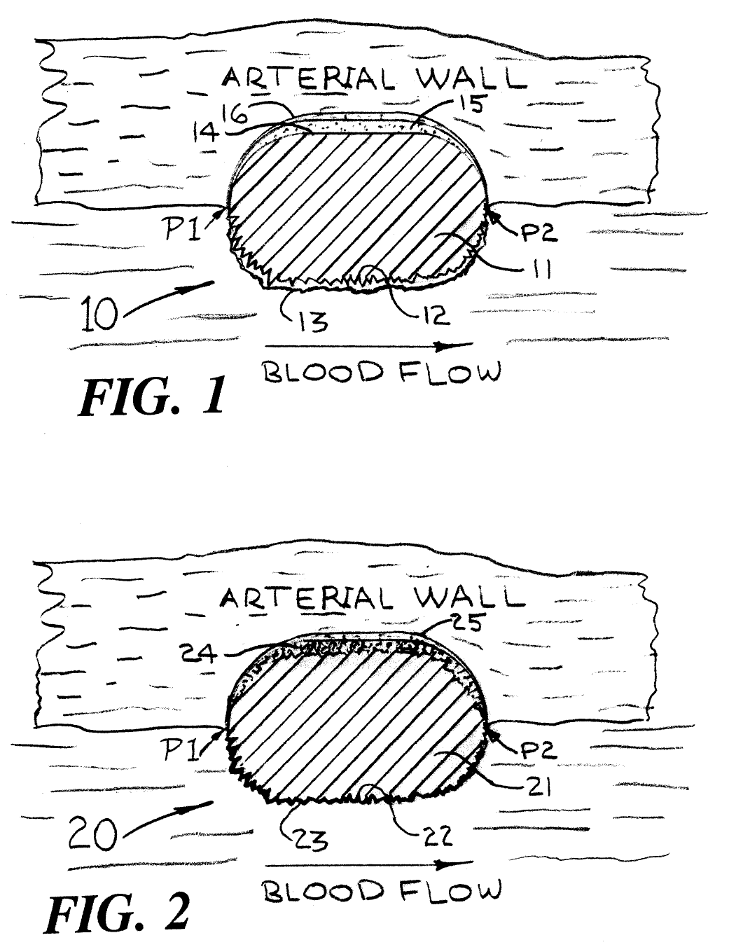

FIG. 1 is a cross section of one strut 10 of a stent (not shown) that has been deployed into an artery that had limited blood flow due to the presence of a stenosis. It should be understood that stents have a multiplicity of such stent struts 10. Some stent struts form a circumferential set of strut members that create a scaffold-like structure to keep the artery open. Other struts are generally used to keep the circumferential set of strut members joined together to form a single stent structure.

The stent strut 10 of FIG. 1 has a metal strength member 11 that may have a roughed surface 12 on the strut's luminal surface and a smooth surface 14 on the strut's abluminal surface. It should be understood that, to a first approximation, P1 and P2 mark the boundaries between the abluminal surface and the luminal surface of the stent strut 10. It should also be understood that the strut may be roughened on both its luminal surface and its abluminal surface.

FIG. 1 shows that the coating 15, which coating contains the anti-restenosis drug, is attached to the abluminal surface 14 of the stent strut 10. It should be understood that the coating 15 could be attached to some small part of the luminal surface of the stent strut 10 or to less than all of the abluminal surface 14. Ideally, the coating 15 would not extend onto the stent's luminal surface beyond the points P1 and P2. The inner coating 15 would typically be a biodegradable coating such as polyester amid (PEA) or its equivalents or a permanent polyester coating such as the combination of EVA and BMA as used on the Cypher stent. Most of the anti-restenosis drug contained within this inner coating 15 would be released into the arterial wall as the coating 15 degrades. It is envisioned to place an outer coating 16 onto the inner coating 15, which coating 16 would ideally be a biodegradable coating that creates a time delay for the release into the arterial wall of the anti-restenosis drug contained in the coating 15. An optimum set of coatings 15 and 16 would both be the biodegradable coating PEA. A typical time delay would be 10±5 days before the drug in the coating 15 starts entering the arterial wall. During that time, the endothelial cells would not be exposed to the anti-mitosis drug so that those cells could proliferate onto the luminal surface coating 13 of the stent strut 10. Although some anti-restenosis drug from the coating 15 might enter the arterial wall by osmosis through the outer coating 16, this minimum release of the anti-restenosis drug would not significantly interfere with the creation of the endothelial cells that are needed to cover the luminal surface coating 13 of the stent strut 10.

It should be understood that there are many variations in the design of the stent strut 10 that are within the scope of the present invention. As an example, the surface 12 of the stent strut 10 could be roughened only on the luminal surface of the stent strut 10 or all surfaces of the metal strength member 11 could be entirely smooth. Also, the luminal surface of the stent strut 10 could be with or without a coating to prevent platelet deposition, or the luminal surface coating 13 could be a porous carbon which has a somewhat roughened surface even if the surface 12 is smooth. Still further, the coating 13 could be an ultra-thin coating of carbon or Hepacoat placed onto the roughened surface 12.

Still further, the coating 15 could contain any type of effective anti-restenosis drug such as Taxol, sirolimus, everolimus or any other drug in the imus family of anti-restenosis drugs. Still further, the coatings 15 and 16 could be of the same type of polymer or its equivalent, or coating 15 could be one type of coating and coating 16 could be of another type. Although for a stent that is implanted in a blood vessel it appears to be desirable to coat every stent strut 10 as shown in FIG. 1, it should be understood that only some of struts of an entire stent could be of a design like the stent strut 10 of FIG. 1.

FIG. 2 is a cross section of a stent strut 20 deployed into the wall of an artery. As with FIG. 1, the surface above the points P1 and P2 is the abluminal surface and the surface below the points P1 and P2 is the luminal surface of the strut 20. The strength member 21 is shown with a roughened luminal surface 22 and a roughened abluminal surface 24. The surface 22 is coated with a very thin layer of an anti-thrombogenic coating 23 such as carbon or Hepacoat. The roughened abluminal surface 24 would contain an anti-restenosis drug such as sirolimus without any polymer. Thus, the shelf life of the anti-restenosis drug would not be compromised by being placed into a polymer. The abluminal surface 24 could be coated with a very thin layer of carbon, but that would not be necessary to provide the desired long shelf life. On top of the roughened abluminal surface 24, a biodegradable coating 25 could be placed that would provide for a delay in the release of the anti-restenosis drug. Thus the endothelial cells could promptly begin to have mitosis to cover the luminal surface coating 23 because the anti-restenosis drug would be delayed in its release and also because there is essentially no anti-restenosis drug on the luminal surface of the stent strut 20. The luminal surface of the strut 20 would be optimized to prevent thrombosis because of four factors, namely: 1) it is roughened surface that encourages endothelial cell deposition and discourages platelet deposition, 2) has an anti-thrombogenic coating such as carbon; and 3) the anti-restenosis drug is not on the strut's luminal surface, and 4) the anti-restenosis drug is delayed in its release because of the coating 25 on the abluminal surface of the strut 20. Thus it is expected that a stent made up of struts 20 as shown in FIG. 2 would be an optimum design to prevent stent thrombosis.

Of considerable importance is a feature of the present invention which is a stent design to decrease the use of an anti-thrombogenic drug like Plavix after implantation of a coronary stent. The stent designs of FIG. 1 or 2 could be used to limit the need for Plavix which would be of great benefit to patients who cannot tolerate such drugs, or who require a later surgical treatment where Plavix could promote excessive bleeding. Still further, the design of the present invention would allow for a significant decrease in health care costs by drastically reducing the need for Plavix after stent implantation. Still further, with the design of the present invention, aspirin alone could be used to prevent restenosis after only a one to three month regimen of the use of Plavix.

Although stents would typically be implanted into a coronary or peripheral artery of a human patient, it should be understood that the stent structures as described herein could also be placed into any blood vessel of a human patient.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. A stent for placement into a blood vessel of a human patient, the stent being formed from a multiplicity of struts with each strut having a luminal surface and an abluminal surface, at least some of the struts having an inner coating that is placed generally onto the abluminal surface of the stent struts, this inner coating including an anti-restenosis drug that can be gradually released to decrease the proliferation of arterial wall cells that tend to be generated when the stent is deployed into the wall of the blood vessel, the inner coating being covered by an outer coating that is a biodegradable coating that does not contain a drug to prevent restenosis of the blood vessel, the function of the outer coating being to delay the release of the anti-restenosis drug from the inner coating so that endothelial cells will more rapidly cover the luminal surface of the stent, the stent struts also having a luminal surface that is generally free from having a coating that includes an anti-restenosis drug.

2. The stent of claim 1 where the luminal surface of at least most of the stent struts is a roughened surface to promote the deposition of endothelial cells.

3. The stent of claim 2 where the roughened surface has a coating that is used to prevent platelet deposition and/or enhance the deposition of endothelial cells.

4. The stent of claim 3 where the coating is either carbon or a heparin based coating.

5. The stent of claim 1 where the luminal surface of at least most of the stent struts is covered by a coating that generally inhibits the adhesion of platelets from the bloodstream.

6. The stent of claim 5 where the coating on the luminal surface of most of the stent struts is a smooth carbon coating.

7. The stent of claim 5 where there is a coating on the luminal surface of the stent that is a porous carbon coating.

8. The stent of claim 5 where the coating on the luminal surface of most of the stent struts is a heparin based coating.

9. The stent of claim 1 where both the coatings on the abluminal surface of the stent struts are of the same material except that the inner coating contains an anti-restenosis drug and the outer coating does not contain an anti-restenosis drug.

10. The stent of claim 1 where the inner coating and the outer coating are formed from different materials.

11. The stent of claim 1 where the outer coating is designed to generally delay the initial release of the anti-restenosis drug contained in the inner coating for approximately 10±5 days.

12. The stent of claim 1 where every strut of the stent has a luminal surface that is free from having a coating that releases an anti-restenosis drug and every stent strut has an inner coating on its abluminal surface that includes an anti-restenosis drug that is covered by an outer coating whose function is to delay the release of the anti-restenosis drug contained in the inner coating from entering the wall of the blood vessel.

13. The stent of claim 12 where the luminal surface of every stent strut is coated with a material that tends to decrease the adhesion of platelets.

14. The stent of claim 13 where the coating on the luminal surface of every stent strut is either carbon or a heparin based material.

15. A stent for placement into a blood vessel of a human patient, the stent being formed from a multiplicity of struts with each strut having a luminal surface and an abluminal surface, at least some of the struts having a roughened abluminal surface with an anti-restenosis drug placed into that roughened surface so that the anti-restenosis drug can be gradually released into the arterial wall to decrease the proliferation of arterial wall cells that tend to be generated when the stent is deployed into the wall of the blood vessel, the roughed surface containing the anti-restenosis drug also having an exterior coating that does not contain an anti-restenosis drug, that exterior coating providing a delay in the release of the anti-restenosis drug into the arterial wall, at least some of the stent struts having a luminal surface that is generally free from having a coating that includes an anti-restenosis drug, the luminal surface also being designed to prevent the deposition of platelet cells and to encourage the deposition of endothelial cells.

16. The stent of claim 15 where the luminal surface is a roughened surface.

17. The stent of claim 15 where the luminal surface is coated with carbon.

18. The stent of claim 15 where the exterior coating of the stent's abluminal surface is a biodegradable coating.

19. A method for allowing patients who have an implanted stent to decrease the length of time during which they are required by their physician to take an anti-thrombogenic drug such as Plavix, the method including the following steps:
a) creating a stent that has two abluminal surface coatings, the inner coating including an anti-restenosis drug and an outer coating that does not contain an anti-restenosis drug, which outer coatings provides for a delay in the release of the anti-restenosis drug into the cells of the arterial wall;
b) placing a coating on the luminal surface of the stent that is anti-thrombogenic; and
c) implanting the stent described in a) and b) into an artery of a human subject.
